(19) United States
(12) Patent Application Publication
(10) Pub. No.: US 2018/0117202 A1
(43) Pub. Date: May 3, 2018

(54) REACTIVATABLE AIR PURIFICATION PAD WITH MOLECULAR SIEVES AND PROCESS

(71) Applicant: Weiming Gao, Toronto (CA)

(72) Inventor: Weiming Gao, Toronto (CA)

(21) Appl. No.: 15/343,188

(22) Filed: Nov. 3, 2016

(51) Int. Cl. A61L 9/014; B01J 20/34; B01J 20/18; B01J 29/90; B01J 38/02; B01D 53/04

(52) U.S. Cl. CPC A61L 9/014; B01J 20/3483; B01J 20/18; B01J 29/90; B01J 38/02; B01D 53/0462; B01D 2259/40088; B01D 2253/106; B01D 2253/104; B01D 2253/25; B01D 2257/708; B01D 2257/90; A61L 2209/22

(57) ABSTRACT

This innovative invention provides a re-activatable Air Purification Pad made of Molecular Sieves/Catalysts, which effectively and efficiently absorbe, disolve and remove the TVOC and odour in the air. This invention can disolve and release the absored TVOC and/or odour when it is heated, which is hence re-activated. This invention, including the product and the process, is effective and efficient in absorbing the TVOC and odour. It is simple in installation and application. It realizes a reduction in tooling and the costs of ongoing maintenance through its re-activation. Moreover, it is safe in use as it is made of heat-resistant materials and doesn't create second polution by its purification embodiments.

### CROSS-REFERENCE TO RELATED APPLICATIONS

Not Applicable

### STATEMENT REGARDING FEDERALLY SPONSORED RESEARCH

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB).

Not Applicable

### STATEMENT REGARDING PRIOR DISCLOSURES BY THE INVENTOR OR A JOINT INVENTOR

Not Applicable

### FIELD OF INVENTION

The invention provides an unprecedented way of low-cost, high-efficiency air purification.

### BACKGROUND OF THE INVENTION

TVOC (Total Volatile Organic Compounds) are the main source of contaminants in the air. The TVOC include polar organic particles, no-polar organic particles and sub-polar organic particles. For quite some time, people have been trying different approaches to clean and remove TVOC in the air. These various approaches are used in existing commercial air purification devices and equipments. These approaches include using activated charcoal, UV light and ozone to absorb and/or dissolve harmful organic particles. However these known approaches are characterized by the following deficiencies (they are numbered here and quoted again later): 1. ineffective and/or inefficient result, 2. produces subsequent second contaminants, 3. other negative side effect, 4. high assembly costs and high running costs, 5. not compatible and/or not scalable structure, 6. not repeatable. These deficiencies and issues remain unsolved.

The activated charcoal as sorbent medium only has limited absorbing capacity for some group of organics. The majority of no-polar organics and sub-polar organics can not be absorbed. For example, dimethyl sulphide, DME, ethylene, butane and methane could not be absorbed effectively by activated charcoal. Furthermore, the activated charcoal can only be used once, and could not be re-activated through heating in normal living environment. The activated charcoals are expensive to use (deficiency number 4 mentioned above), have limited absorbing capacity hence low efficient result (deficiency number 1 mentioned above). In addition, the current known design structure is not convenient, not compatible and not scalable (deficiency number 5 mentioned above).

UV light can dissolve some organic particles with low molecular weight and relatively simple structure. It can help to purify the air to some extent. However, the dissolution of the organics by UV light can be uncertain and incomplete. The toxin from the dissolution is difficult to estimate and understand. Therefore the risk is quite high. Furthermore the solid organic subjects, such as the painting on the furniture, will be oxidized and damaged when exposed under the UV light. In addition, the UV light in working will generate ozone. The accumulation of ozone within the room is harmful to human being, animals and plants. Prolonged exposure to ozone is extremely harmful to human health. There is control level and/or recommended safe level for the ozone in most countries. Thus, the UV light approach is not efficient (deficiency number 1 mentioned above), has subsequent second contaminant (deficiency number 2 mentioned above), has negative side effect (deficiency number 3 mentioned above), and is not safe for long-term repeated usage due to the side effect (deficiency number 6 mentioned above).

Ozone has very strong oxidization ability. It can react with a lot of organics. However the resulting products of the reaction vary with the density of the ozone, the humidity of the air, the temperature and the duration of the reaction time. There are lots of interim products, which could have high-risk toxins. The ozone exceeding certain thickness threshold is harmful to human being, animals and plants. The ozone will have negative impact on people's health if it is used repeatedly. Hence the ozone approach has the following deficiencies: produces subsequent second contaminant (deficiency number 2 mentioned above), negative side effect (deficiency number 3 mentioned above), and is not safe to long term repeated usage due to the side effect (deficiency number 6 mentioned above).

The Air Purification Pad invention has solved all issues and deficiencies in the above mentioned approaches through an innovatively fabrication and a ground-breaking methodology. The invention provides numerous benefits and advantages over the known approaches. This invention utilizes Molecular Sieves (specially designed for high absorption) made of high surface ratio AL203/SIO2 chemical composite and/or catalysts made of these Molecular Sieves as sorbent medium, coupled with hollow ceramic particles to enhance airflow. A metal frame is designed to host the mixture of Molecular Sieves/catalysts and hollow ceramic to form the Air Purification Pad (Hereafter called Air Pad). The Air Pad is the same or can be easily customized to the measure as the outlet register or return registers of the HVAC ducting. Alternatively, the Air Pad can be built to fit with the dimensional measures of the filter frame in the HVAC machine or can be designed and/or assembled to have the dimensions of any specific air pathway. This design ensures the ease of assembling or de-assembling of the Air Pad for re-activation of the molecular Sieves/Catalysts.

The Air Pad has excellent absorbing capacity for volatile organic vapour in normal atmosphere contained within an enclosed space, like indoor, mobile vehicles. The Air Pad works well on absorbing both no-polar and polar volatile organic vapour. The Air Pad absorbs the TVOC and odour in the airflow passing through the pad via the HVAC ducting, or any forced airflow passing through the pad. Thereby, the TVOC and odour in the airflow are absorbed by the sorbent medium, thus the TVOC and odour pollution in the enclosed space (including indoor, mobile vehicles) air is reduced progressively. When the sorbent medium approaches the absorbing capacity saturation, or after reaching recommended duration of use, the Air Pad can be de-assembled and be placed on the top of heating instruments with any exhaust device overhead. The heating will release the TVOC and/or odours absorbed by the Molecular Sieves, or speed up the oxidative decomposition of the TVOC. At this moment, the exhaust device expels the released TVOC/odour or decompositions to outdoor. The sorbent medium and the catalysts are re-activated through this heating and releasing cycle. After this heating and re-activation cycle, the Air Pad can be assembled and put back to function in HVAC ducting and/or the filter frame of the HVAC machine. Hence, the Air Pad is in function for another new cycle of the TVOC absorbing process to purify the air in an enclosed space (including indoor and mobile vehicles). The Air Pad can be re-activated, and reuse for repeated cycles. It is easy to assemble in place, convenient to use, high effective and efficient in TVOC/odour absorbing, reliable in use, and low cost in operation. There is no need for replacement of the parts.

With the above, this invention provides an unprecedented way of low-cost, high-efficiency air purification.

### DESCRIPTION OF PRIOR AT

It is known in the prior acts that various means and methods are generally disclosed to remove odors from indoor air.

For example, Canadian Patent Number 1259295, which issued to STEPHEN E FRAZIER on Sep. 12, 1989, discloses an air filter element for removing odours from indoor air having the mixture of activated carbon. The described element is expensive to use (4), have limited absorbing capacity hence low efficient result (1).

The Canadian Patent Number 28184444, invented by ANDREY V. LIVCHAK and RICK A. BAGWELL, filed on Nov. 18, 2011 is in the stage of examination requested. This patent describes a device and method for air purification with UV light source.

Similarly, the Canadian Patent 1108068, which issued to MANFRED R. Burger on Sep. 1, 1978, describes the air filter with activated charcoal and electricity.

However these known approaches are characterized by the following deficiencies: 1. ineffective and/or inefficient result, 2. produces subsequent second contaminants, 3. other negative side effect, 4. high assembly costs and high running costs, 5. not compatible and/or not scalable structure, 6. not repeatable. These deficiencies and issues remain unsolved

### BRIEF SUMMARY OF THE INVENTION

This invention uses Molecular Sieves or active oxidation catalysts made of porous AL203/SIO2 with high ratio of surface area as sorbent medium. The porousness, high surface ratio and different ratio of SI/AL enable the Molecular Sieves and catalysts to absorb various organics and odour, therefore the sorbent medium used in this invention has very broad spectrum of absorbing ability and capacity. In particular, in handling no-polar organic pollution including odour, Molecular Sieves with high ratio of silicon to aluminium, which is hydrophobic, will have excellent absorbing effect.

The Molecular Sieves can also be loaded or mixed with activating components such as rare earth oxidation, which include Ce2O3, CeO2, La2O3 , other rare earth oxidation and platinum family metals. The platinum family metals include Pt, Pd, Os, Jr, Ru, Rh and other metals. The activating components can also be made of Copper oxide (CuO). These activating components have excellent oxidative and catalytic ability and capacity when heated in high temperature. The Molecular Sieves are transformed into active oxidation catalysts after being loaded with activating components.

When the sorbent medium reaches to the saturation capacity of absorbing or after reaching recommended duration of use, the Air Pad hosting sorbent medium is placed onto a heating device for re-activation. The heating device is in a location with an exhaust device overhead to expel the released air outdoor. Then the TVOC absorbed will be discomposed effectively by the catalysts inside the sorbent medium when heated. In the case where the catalysts are not used, the TVOC and/or odour absorbed will be released. Both released TVOC/odour and decomposed TVOC/odour will be expelled to outdoor through the vent over the heating device. Hence the contamination in an enclosed space (including indoor and mobile vehicles) are cleaned and removed. In the meantime, the sorbent medium is re-activated after heated and releasing the TVOC/odours absorbed. The Air Pad with the re-activated sorbent medium can be put back into next absorbing working cycle.

This invention provides numerous benefits and advantages over known air purification methods. In particular, the sorbent medium in the Air Pad can be used repeatedly .This unique “heated then reactivated and reused” feature differentiates the absorbing base from traditional activated charcoal. The activated charcoal can only absorb the polar organics in gaseous state, and has very limited absorbing power to no-polar organics in gaseous state. Furthermore, the activated charcoal can not be re-activated through high temperature under normal air environment.

In addition to the foregoing attributes, a significant advantage of this invention is in removing the toxic air from indoor/inside to outdoor/outside. The process does not create second pollution.

The sorbent medium of this invention are made from materials with low pile-up density, in the shape of granule, stripe, or the any other shape with hollow structure. These shapes and structures allow for optimal airflow. Furthermore, hollow ceramic granule/stripe and irregular ceramic granule/stripe like raschig rings can be mixed into the sorbent medium. The addition of the ceramic material (with feature of heat resistance and flame resistance) improves the airflow and reduces the air resistance. It also enhances the exposure of the sorbent medium to the toxic material in the air and enhances the interaction between them.

This invention is also designed with mechanical features of the flexibility to be mounted on the HVAC ducting, or to be assembled for any specific air pathway. This feature liberates it from specialized installation and maintenance. It also provides a convenient way of reactivating the sorbent medium. The sorbent medium mixture as described above is filled into the heat-resistant metal screen frame box to fabricate the Air Pad. The air will pass through this metal screen frame box filled with sorbent medium mixtures—Air Pad before going into/or getting out of the HVAC ducting. In the case that without a HVAC ducting, forced airflow can be directed to pass through the Air Pad. This Air Pad has the feature of heat-resistant, flame-resistance, it can stand the heating treatment by various heating devices, like oven, toast oven or even gas heating device. This Air Pad can maintain its completeness of the structure and physical shape over the repeated heating treatment.

The Air Pad can be designed to have the same dimensions as the inside dimensions of the air filter, and/or the inside dimensions of the inhale/exhale registers in the forced air HVAC ducting in both residential and commercial buildings. Hence the users can attach the Air Pad to the HVAC ducting easily and directly. The users can also remove the Air Pad easily and directly and re-install them back after the Air Pads is re-activated. The Air Pad is re-activated by heating treatment.

The heating treatment equipments used for activating the sorbent medium—Molecular Sieves/Catalysts in this invention can be the heating appliance in the kitchen. They can be any heating devices, like electronic oven, toast oven, or gas oven, and propane oven; as long as there is an exhaust vent overhead to expel the released air. The toxic material released and decomposed during the re-activating process can be expelled outdoor through the kitchen exhaust fan. Hence the users do not need extra investment for the heating and venting equipment. This Air Purification Pad is convenient to use, runs with low operating cost, has high effectiveness and efficiency, is reliable, removes toxic material completely and does not create second pollution. This invention—Air Purification Pad is superior to other kind of air purification approach/equipments.

### BRIEF DESCRIPTION OF THE SEVERAL VIEWS OF THE DRAWINGS

FIG. 1A, FIG. 1B and FIG. 1C provide the component list of the materials used. They include ceramic particles, molecular sieves/catalysts, mixture of molecular Sieves, and catalysts with ceramic particles, metal profile and stainless steel sieves.

FIG. 2 is a demonstration of assembling an Air Purification Pad.

FIG. 3 is an enlarged view of the Air Purification Pad.

FIG. 4A and FIG. 4B show an example of uasge of Air Pad in HVAC ducting on floor or ceiling.

FIG. 5A, FIG. 5B and FIG. 5C show an example of uasge of Air Purification Pad in HVAC ducting on wall.

FIG. 6 is an illustration of the process for re-activating the Air Purification Pad.

FIG. 7 lists the legend of the drawing reference numbers.

### DETAILED DESCRIPTION OF THE INVENTION

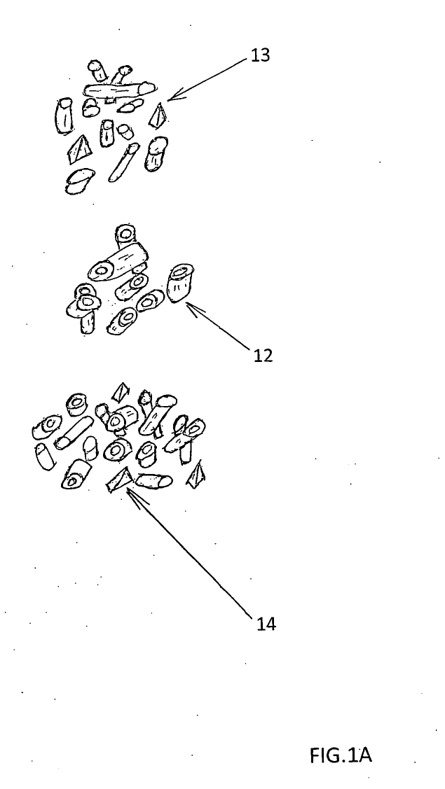

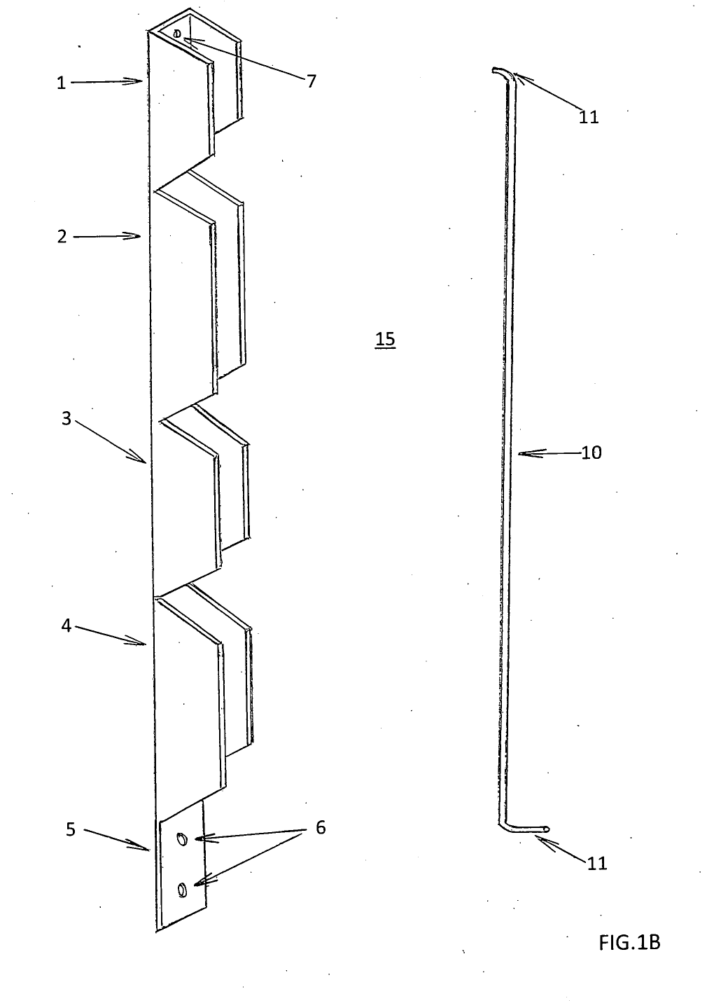

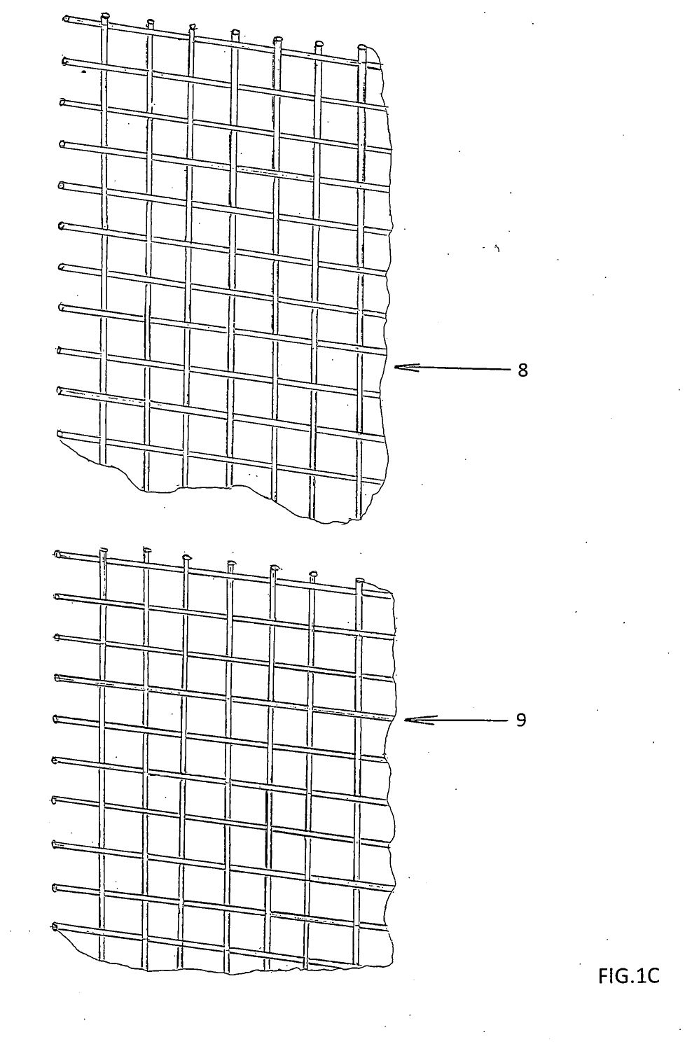

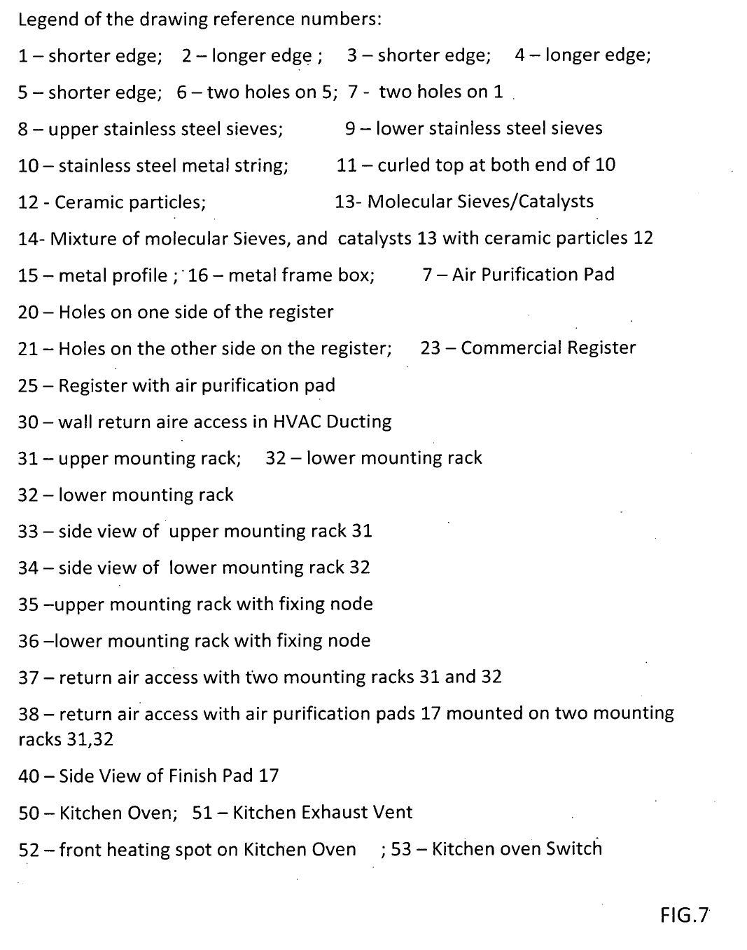

With reference to the drawings, and in particular, with reference to FIG. 1A to FIG. 7, the Air Purification Pad comprises of a framed heat-resistant metal box, with upper surface and bottom surface made of metal sieve screen. This box is filled with Molecular Sieves made of high surface ratio AL203/SIO2 chemical composite, or with active oxidation catalysts made of Molecular Sieves loaded with activating components. The Molecular Sieves and/or catalysts are mixed with hollow ceramic particles. This Air Purification Pad can be customized to the dimensions compatible to required air pathway.

FIG. 1A, FIG. 1B and FIG. 1C provide the component list of the materials used. These materials are selected to produce the Air Purification Pad.

Molecular Sieves 13 are molecular sieves or active oxidation catalysts made from porous AL203/SIO2 with high ratio of surface area. They can be in granule, round, spherical, stripe, irregular shape, or any other shape with hollow structure. The molecular sieves or active oxidation catalysts can be specifically designed and selected based on the target environment. The Molecular Sieves preferably used can be Molecular Sieves ZSM5. The catalysts preferred is ZSM5 loaded with activating components. The sorbent medium refers to either molecular sieves or catalysts, or the mixture of both.

Ceramic particles 12 are hollow ceramic particles. They can be in granule, round, spherical, stripe like raschig rings, irregular shape, or any other shape with hollow structure. These Ceramic particles 12 are mixed into Molecular Sieves/Catalysts 13. The addition of the ceramic material (with feature of heat resistance and flame resistance) improves the airflow and reduces the air resistance. It also enhances the exposure of the sorbent medium to the toxic material in the air and enhances the interaction between them.

Mixture of the sorbent medium and the Ceramic particles 14 is the mixture of Molecular Sieves, and catalysts 13 with ceramic particles 12. The ratio of the mixture can be from about 0 to 100 weight percentage of Molecular Sieves, from about 0 to 100 weight percentage of active oxidation catalysts, and from about 0 to 95 weight percentage of ceramic particles. In the preferred embodiment, when Molecular Sieves/catalysts ZSM5 is used, the ratio is 50 percentage weight of ZSM5, 50 percentage weights of ceramic particles.

The Air Pad can be customized to the required dimensions of the airflow pathway. In this preferred embodiment, the dimensions of a commercial register for HVAC ducting are used as an exmaple.

The metal profile 15 is a metal profile, which is heat-resistant and flame-resistant. It is used to fold into a retangular frame. 1, 3, and 5 are the shorter edges. 2 and 4 are the longer edges. 5 covers 1 when they are folded together. Two holes 6 are prepared on 5. Two holes 7 are prepared on 1. 6 and 7 are reconciled when 1 and 5 are folded together. Two rivets are used to put 6 and 7 together. In the preferred embodiment, aluminun profile is used.

Metal string 10 is stainless steel metal string. 11 is the curled top at both ends of the string. Metal string 10 is used as a stopper.

Stainless steel seives 8 and 9 are stainless steel seives , heat-resistant and flame-resistant. They are used to fit into the metal frame folded by 15, and become the upper stainless steel sieves surface and lower stainless steel sieves surface of a metal screen box.

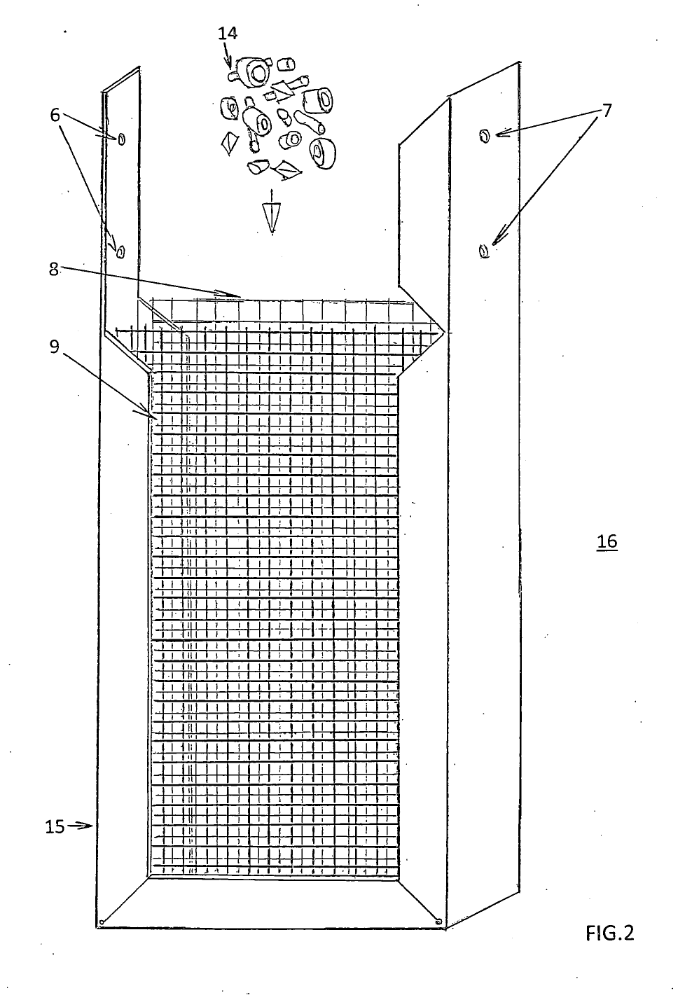

FIG. 2 is a demonstration of assembling an Air Purification Pad.

Use metal profile 15 to fold into a retangular frame, with 1 and 5 open and pending to close. Slides 8 and 9 into both side of the frame. This is the metal frame box 16 ,with an open end. Fill the mixture 14 into this metal box. Close 1 and 5 together and put rivet on holes 6 and holes 7. Thus the finsihed product 17—the Air Purification Pad is assembled.

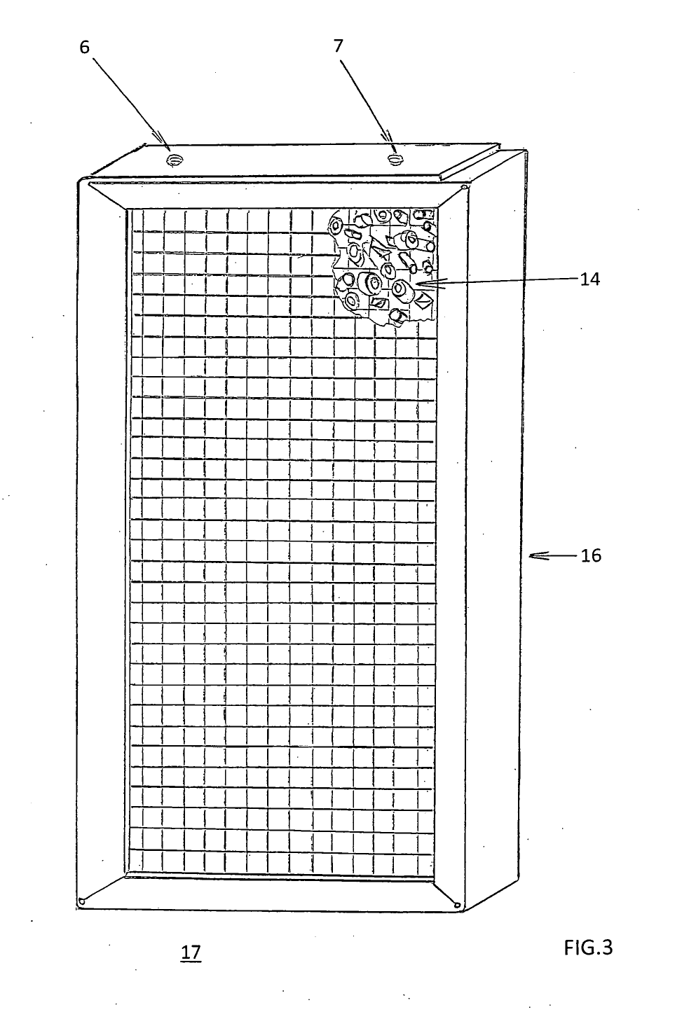

FIG. 3 is an enlarged view of the Air Purification Pad 17.

From this enlarge drawing, it is observed that between the two screens 10 and 11 are the sorbent medium and ceramic particle 14. The air passes through one of the screens 10 or 11, then interact with sorbent medium. The TVOC and odour in the air is absorbed by sorbent medium. Then the air is cleaned and flows out through screens of 10 or 11. This shows how the air is purified.

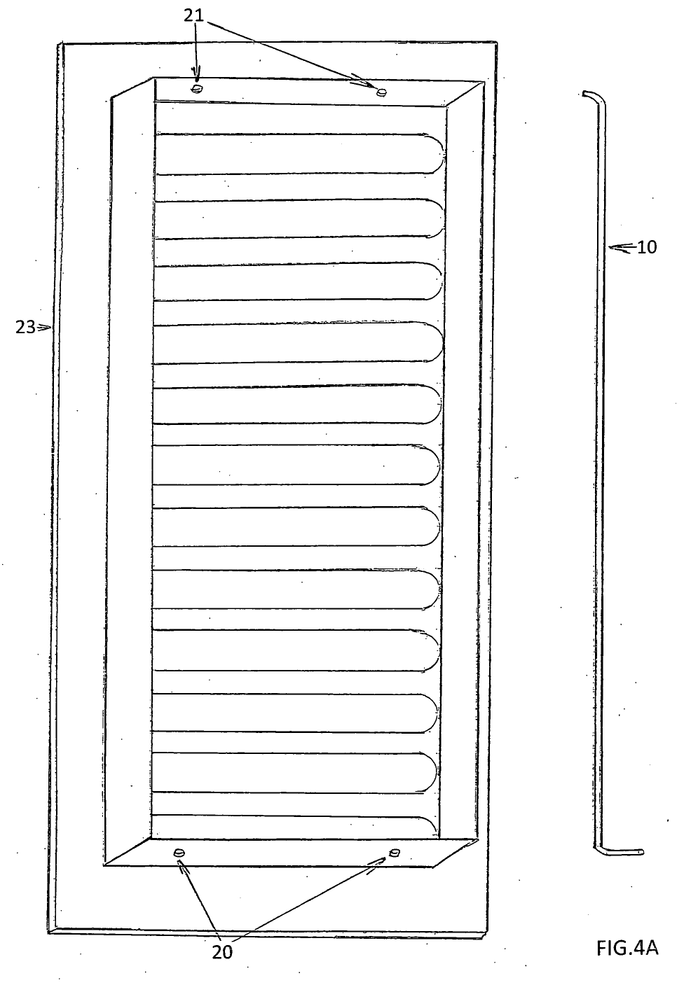

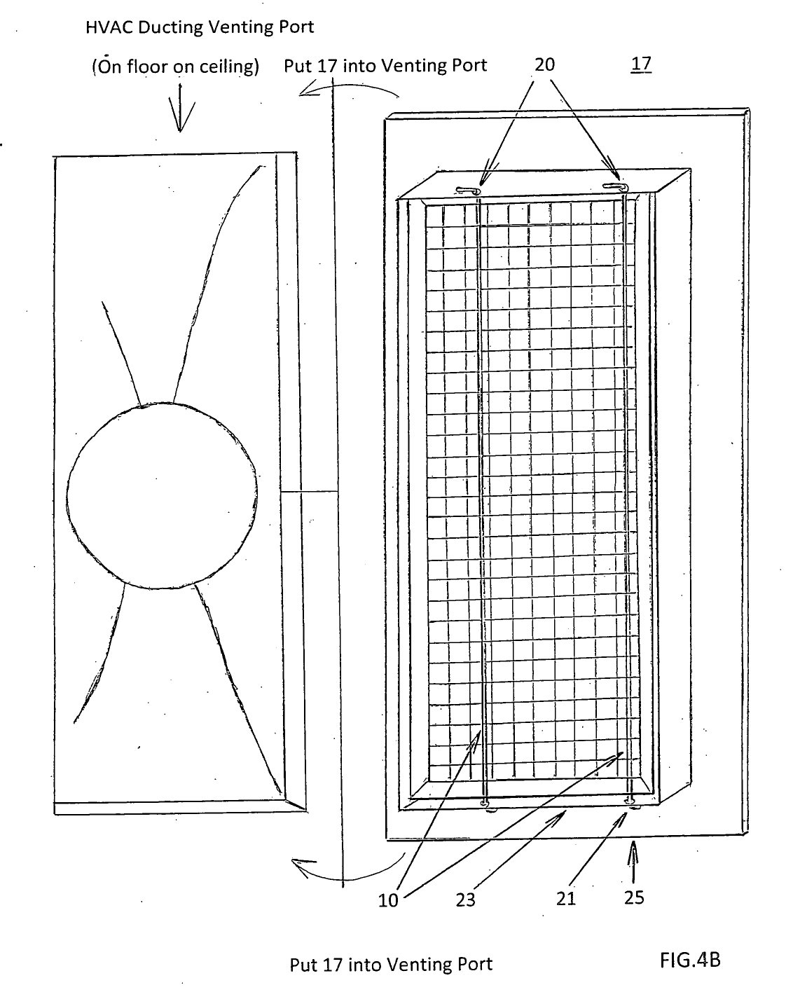

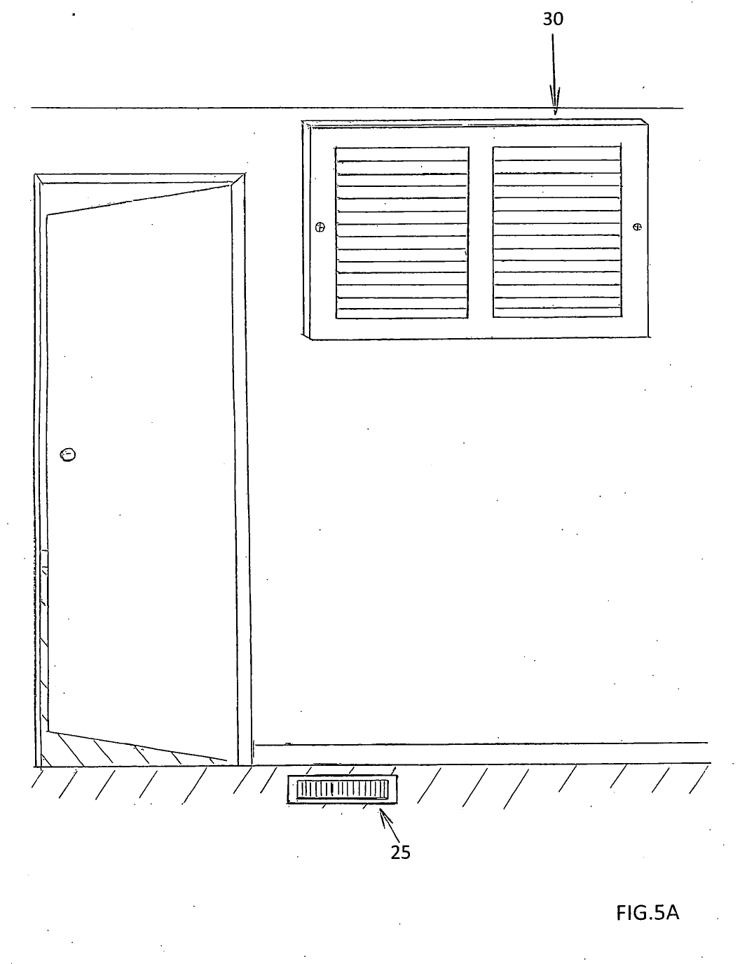

FIG. 4A and FIG. 4B show an example of usage of an Air Purification Pad in HVAC ducting on floor or ceiling.

23 is a commercial register used in HVAC ducting. Holes 20 , 21 are used for stopper string 10. Now the buffer flip is removed.

10 is the stainless string stopper in FIG. 1B.

17 is the finished Air Purification Pad fabricated in FIG. 3 , with the cutomized dimensions of the inside measure of register 23.

Slide and place Air Pad 17 into the inside of 23, put both stainless strings 10 cross the holes 20 and 21. These two strings serve as stopper to keep the Air Pad inside the register. This will finish the assembling of the Air Pad into the register 23. This register with Air Purification Pad 25 can be put into the HVAC Ducting, either on the floor or on the ceiling , as you normally would use a register. When the HAVC is in working, the airflow passes through the Air Pad, the TVOC and/or odour is captured by the sorbent medium inside the Air Pad progressively. Hence the air in an enclosed space (including indoor and mobile vehicles) is cleaned and purified.

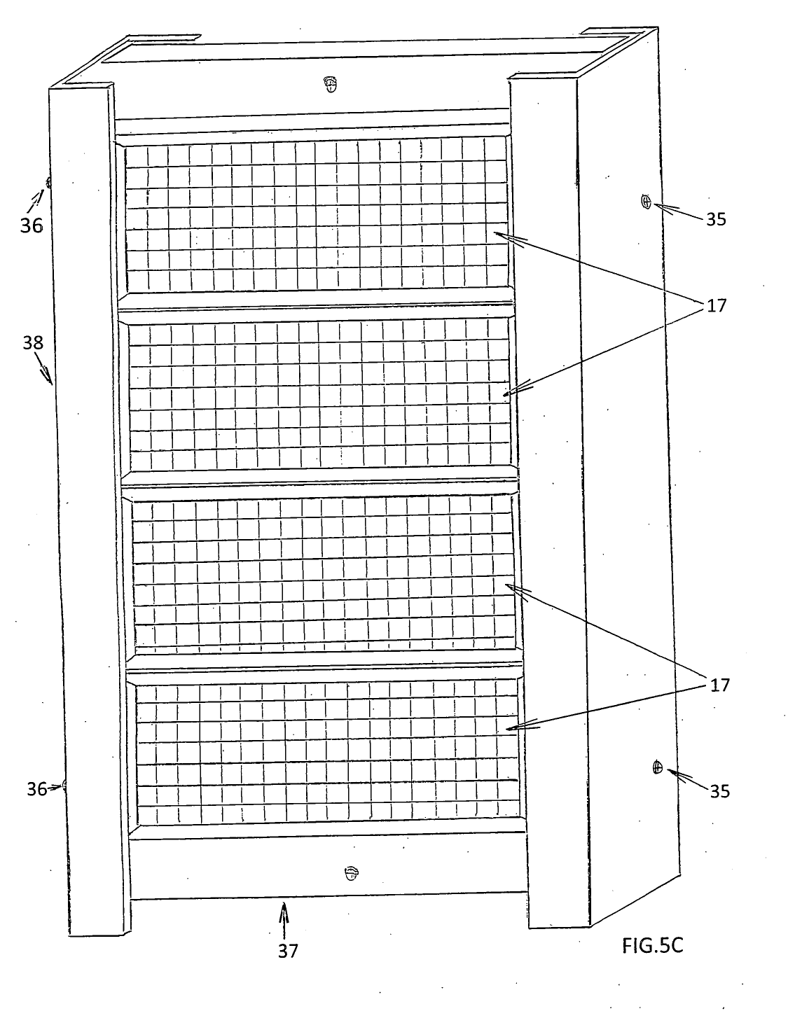

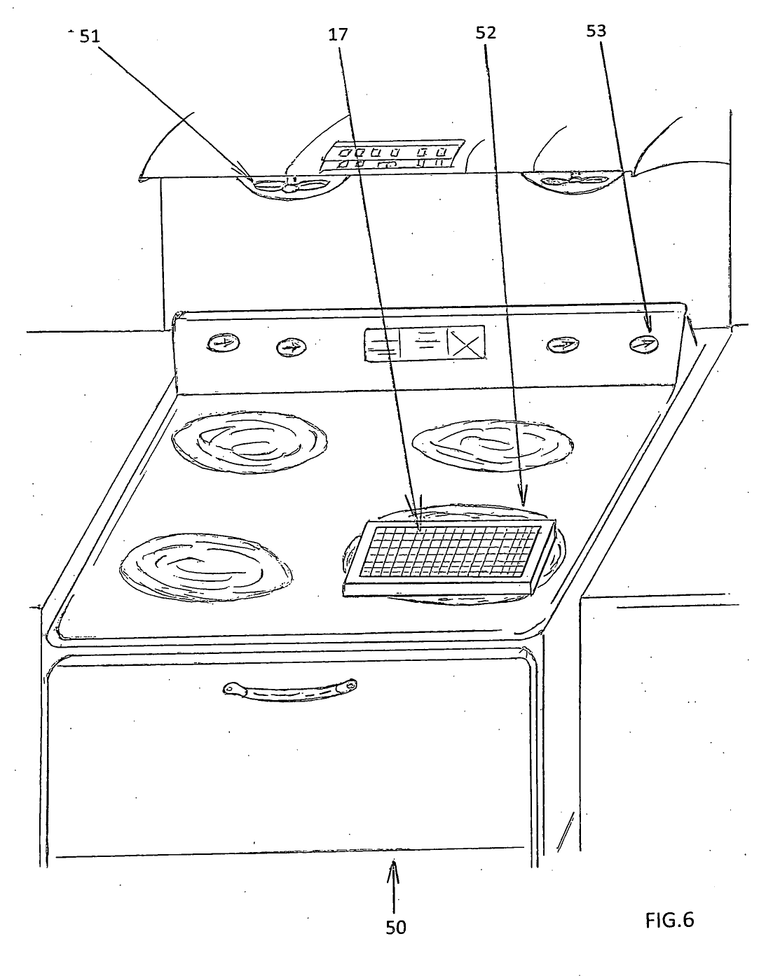

When the Air Pad 17 stays in the register 23 for a recommended duration, 17 can be removed from the register easily. Just remove the two stainless steel strings 10 from the register 23, slide out the Air Pad 17. Then, the Air Pad can be put through the reactivation process, as shown in FIG. 6.

After the reactivation process in FIG. 6, the Air Pad can be put back in the register, added on the stopper 10. The Air Pad in the register now is ready for purification function again.

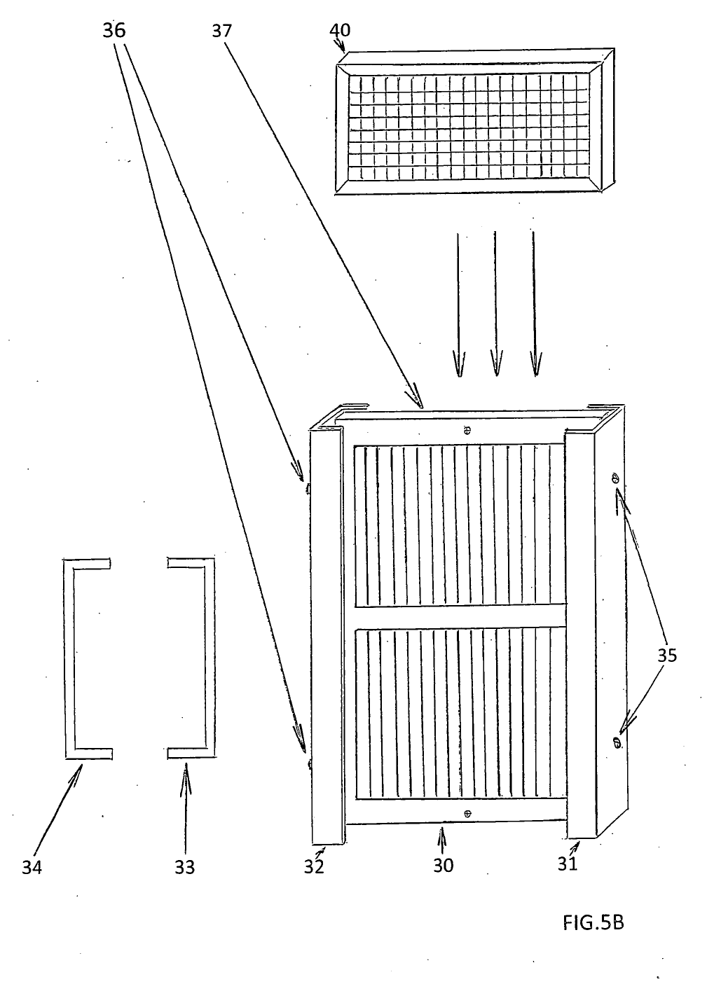

FIG. 5A, FIG. 5B and FIG. 5C show an example of usage of Air Purification Pad 17 in HVAC ducting on the wall.

This figure is to demonstrate the Air Pad's mechanical flexibility and scalability to fit into any required airflow pathway.

30 is the snapshot of the image of the wall return air access in HVAC ducting. It normally has three times the size of the floor register. In this case, an Air Pad can be made to the size of the return air access 30. Alternatively, the combined Air Pad can be assembled by fabricating three preferred standard Air Pads 17 together, as demonstrated in the following paragraph.

31 and 32 are two identical mounting racks. They are used to hold multiple Air Pads. 33 is the side view of rack 31. 34 is the side view of 32. 40 is the side view of 17. The Air Pads can be easily slided into the racks when they are properly positioned.

37 is the snapshot of the return air access, with the two mounting racks 31, 32 fixed on the upper side and the lower side of 30, noted as 35 and 36. The distance between 35 and 36 is properly spaced such that the Air Pads can be easily slided into and between 35 and 36 .The Air Pads are held safely and reliably.

38 is the snapshot of the return air access, with Air Pads mounted on racks 35 and 36. When the HVAC is in working, the airflow passes through the Air Pads, the TVOC and/or odour is captured by the sorbent medium inside the Air Pad progressively. Hence the air in an enclosed space (including indoor and mobile vehicles) is cleaned and purified

When the Air Pads 17 stay on the racks 35 and 36 for a recommended duration, they can be removed from the racks easily by sliding them out. Then, the Air Pads 17 are ready for the reactivation process, as shown in FIG. 6.

After the reactivation process in FIG. 6, the Air Pads can be slided back into racks 35 and 36. The Air Pads in the racks are ready for the purification function again.

similarly, the Air Purification Pad 17 can be customized to fit to the HVAC filter in mobile vehicles.

FIG. 6 is an illustration of the process for re-activating the Air Purification Pad 17.

The heating treatment equipments used for activating the sorbent medium (Molecular Sieves and/or Catalysts) in this invention can be any heating devices, like electronic oven, toast oven, gas oven, or propane oven; as long as there is an exhaust vent overhead to expel the released air.

In this preferred embodiment, the kitchen oven and the overhead kitchen exhaust fan are used. Hence the users do not need extra investment for the heating and venting equipment.

50 is the snapshot of a kitchen oven. 52 is the front pot heating spot. 53 is the switch.

51 is the snapshot of the kitchen exhaust vent over the cooking oven.

Turn on the vent 51; place the Air Pad 17 on heating spot 52, one of the heating spots of the oven. The Air Pad is removed from register or return air access as shown in FIG. 4B and FIG. 5C.

Turn on the heat to maximum by turning the switch 53 on the oven.

The reactivation process starts when the heat is on. The Molecular Sieves and/or Catalysts inside the Air Pad 17 dissolve and/or release the absorbed TVOC and/or odour. The overhead vent 51 expels the released air from the Air Pad to outdoor. The preferred heating time is about 10 to 15 minutes. Molecular Sieves and/or Catalysts 13 inside the Air Pad are reactivated. Turn off the oven; let the Air Pad 17 cool down. The Air Pad 17 is reactivated and can be put back to register or any original application for the next cycle of air purification.

What is claimed are defined as follows:

1. The invention re-activatable and re-usable Air Purification Pad ,which absorbs TVOC and odour in normal enviroment in an enclosed space (including indoor and mobile vehicles), disolves and releases TVOC and odour, thereafter is reactivated when heated, comprises of Molecular Sieves and/or active oxidation catalysts made of porous AL203/SIO2 with high ratio of surface area as sorbent medium, a metal framed box made of heat-resistant metal with upper surface and bottom surface made of metal sieve screen, hollow ceramic particles mixed into Molecular Sieves and/or active oxidation catalysts.
The molecular sieves of porous AL203/SIO2 with high ratio of surface area, with different ratio of SI/AL, can be in the shape of granule, stripe, cylindrical, irregular, or any other shape with hollow structure. These shapes and structures allow for optimal airflow.
The active oxidation catalysts is the said Molecular Sieves loaded or mixed with activating components such as rare earth oxidation, which include Ce2O3, CeO2,La2O3, other rare earth oxidation and platinum family metals. The platinum family metals include Pt, Pd, Os, Ir, Ru, Rh and other metals. The activating components can also be made of Copper oxide (CuO).
Ceramic particles in the shape of hollow, granule, stripe and irregular ceramic granule/stripe like raschig rings are mixed into the molecular sieves and/or the catalysts. These shapes and structures allow for optimal airflow. The adding of ceramic particles further enhances the airflow and maximizes the interaction between the TVOC and odour in the airflow with the Molecular Sieves and Catalysts.
The mixtures of Molecular Sieves and/or Catalysts with ceramic particles are filled into the metal framed box to fabricate the Air Purification Pad.

2. The Air Purification Pad cited in claim 1 wherein Molecular Sieves and/or Catalysts are selected based on the targeted market and the designed function of the Air Purification Pad. Molecular Sieves and Catalysts can be the used combined or solely.

3. The Air Purification Pad cited in claim 1 wherein Molecular Sieves and/or Catalysts can be made of different ratio of SI/AL enabling the Molecular Sieves and catalysts to absorb various organics and odour. For example, in handling no-polar organic pollution including odour, Molecular Sieves with high ratio of silicon to aluminium, which is hydrophobic, have excellent absorbing effect.

4. The Air Purification Pad cited in claim 1 wherein the ceramic particles are heat resistant and flame resistant. They are hollow ceramic granule/stripe and irregular ceramic granule/stripe like raschig rings. These shapes and structures allow for optimal airflow. The adding of ceramic particles further enhances the airflow and maximizes the interaction between the TVOC and odour in the airflow with the Molecular Sieves and Catalysts.

5. The Air Purification Pad cited in claim 1 wherein the metal framed box is made of heat-resistant and flame-resistant metal. The upper surface and bottom surface are made of metal sieve screen, hence the airflow can pass through the screen, then the said mixture of Molecular Sieves and/or Catalysts mixed with ceramic particles. The metal framed box maintains its shape and structure to hold the said mixture of Molecular Sieves and/or Catalysts and ceramic particles under high temperature.

6. The Air Purification Pad cited in claim 1 wherein the mixtures of Molecular Sieves and/or Catalysts with ceramic particles can have different ratio of Molecular Sieves/Catalysts to ceramic particles. The ratio can vary from 100 to 0 weight percentage to 5 to 95 weight percentage. The consideration and decision of the ratio depends on the shape of both Molecular Sieves/Catalysts and ceramic particles. The ratio is chosen to ensure the optimal airflow and maximum interaction between TVOC and odour in the airflow with the Molecular Sieves/Catalysts. Targeted market and function from economic consideration is also a factor in selecting the ratio.

7. The Air Purification Pad cited in claim 1 wherein the mixtures of Molecular Sieves and/or Catalysts with ceramic particles comprise 0 to 100 weight percentage of Molecular Sieves, 0 to 100 weight percentage of active oxidation catalysts, and 0 to 95 weight percentage of ceramic particles.

8. The Air Purification Pad cited in claim 1 wherein the metal framed box is customized to the required dimensions of any airflow pathway. The dimensions can be the inside dimentions of the conventional registers, or the dimensions of the air filter frame of the HVAC machine indoor or in mobile vechieles, or required dimensions of any airflow pathway.

9. The Air Purification Pad cited in claim 1 wherein the metal framed box can be made to the preferred dimensions as the inside dimentions of the conventional registers. Then multiple Air Purificaton Pads can be assembled to form new dimesions fitting into the required airflow pathway. This enables the usage of the Air Purification Pad scalable.

10. The Air Purification Pad cited in claim 1 wherein the Molecular Sieves and/or catalysts absorb the TVOC and odour in the air when the airflow passes through the Air Purification Pad. Thus the TVOC and odour pollution in the enclosed space (including indoor and mobile vehicles) is reduced progressively. When the Molecular Sieves and/or catalysts approaches the absorbing capacity saturation, or after reaching recommended time duration of use, the said Air Purification Pad can be placed on the top of heating devices with any exhaust device overhead. The Molecular Sieves/catalysts under heating will dissolve and release the TVOC and/or odours absorbed by the Molecular Sieves/catalysts, or speed up the oxidative decomposition of the TVOC. At this moment, the exhaust device will expel the released TVOC/odour or decompositions to outdoor. The Molecular Sieves and/or catalysts are re-activated through this heating and releasing cycle. After this heating and re-activation cycle, the said Air Purification Pad is re-activated and can be put back to function in HVAC ducting and/or the filter frame of the HVAC machine. Air Pad is in function for another new cycle of the TVOC and odour absorbing process to purify the air. The Air Pad can be re-activated, and reuse for repeated cycles. In the case where there is no HVAC ducting and no HVAC filter, the Air Purification Pad can be used where a directed airflow is created to pass through the Air Purification Pad. For example, a fan blows the air through the Air Purification Pad, which absorbs the TVOC in the air passing through;
hence the TVOC and odour pollution in the enclosed space (including indoor and mobile vehicles) is reduced progressively.

11. The metal framed box in claim 5 is featured with heat resistance and flame resistance. Hence it can be placed on the top of any heating device.

12. The Air Purification Pad cited in claim 1 and claim 10 is featured with heat resistance and flame resistance, fabricated from the metal framed box in claim 5, Molecular Sieves and/or Catalysts in claim 2, and ceramic particles in claim 4. It can be place on the top of heating device for heating.

13. The heating and re-activation process in claim 10 wherein the heating device used for activating the Molecular Sieves/Catalysts in claim 1 can be the any heating devices, even heating appliance in the kitchen. They can be any heating devices, like electronic oven, toast oven, gas oven, or propane oven; as long as there is an exhaust vent overhead to expel the released air.

14. The heating and re-activation process in claim 10 wherein the exhaust vent can be any type of exhaust vent. They can be the exhaust vent in the kitchen.

15. The heating and re-activation process in claim 10 wherein the heating device and the exhaust vent can be the kitchen oven and the kitchen exhaust vent readily available. Hence the users do not need extra investment for the heating and venting devices.

16. The heating and re-activation process in claim 10 wherein Molecular Sieves and/or catalysts under heating dissolve and release the TVOC and odour absorbed. Hence the Molecular Sieves and/or catalysts are re-activated after the said heating process in claim 10. The Air Purification Pad is re-activated and ready for use again.

17. The re-activation of the Molecular Sieves and/or catalysts and the Air Purification Pad is repeatable; hence the Air Purification Pad is re-activatable and re-usable.

18. The heating and re-activation process in claim 10 wherein Molecular Sieves and/or catalysts dissolve and release the TVOC and odour absorbed. The released TVOC/odour and the decomposition of the TVOC and odour are expelled outdoor. The Air Purification Pad is reactivated and put back to function for air purification. Through the absorbing, heating, dissolve and release, re-activation, absorbing cycles, the air in an enclosed space (including indoor and mobile vehicles) is purified progressively and continuously.

19. The heating and re-activation process in claim 10 wherein the metal framed box can be the shape as described in claim 5. The metal framed box can also be extended to a metal container made of the heat-resistant metal, and can be any shape of metal container made of metal sieve when applying the heating and re-activation process in claim 10. The metal container making of heat-resistant metal can be place on the top of heating instrument to re-activate the Molecular Sieves/catalysts inside the container.

20. The claim 8 enables the Air Purification Pad cited in claim 1 to be customized to the required dimensions of any airflow pathway. The dimensions can be the inside dimentions of the conventional registers, or the dimensions of the air filter frame of the HVAC machine, or required dimensions of any airflow pathway. This mechanical flexibility enables easy installation and desembling of the Air Purification Pad for re-activation and then reinstallation. This Air Purification Pad is convenient to use, runs with low operating cost, has high effectiveness and efficiency, is reliable, removes toxic material completely and does not create second pollution.
